(19) United States
(12) Patent Application Publication
(10) Pub. No.: US 2003/0158123 A1
(43) Pub. Date: Aug. 21, 2003

(54) Purified material having size and growth inhibitory effects on cells and tissues

(76) Inventor: John Ernest Hart, Hampshire (GB)

Correspondence Address: SALIWANCHIK LLOYD & SALIWANCHIK, A PROFESSIONAL ASSOCIATION, 2421 N.W. 41ST STREET, SUITE A-1, GAINESVILLE, FL, 326066669

(21) Appl. No.: 10/297,548

(22) PCT Filed: Jun. 8, 2001
(86) PCT No.: PCT/GB01/02552

(30) Foreign Application Priority Data
Jun. 8, 2000 (GB) 0014029.3

(51) Int. Cl. A61K 39/395; A61K 38/17

(52) U.S. Cl. 514/21; 424/130.1

(57) ABSTRACT

A method of purifying a material (“Micrin”) with the ability to reduce cell, tissue and organ size, mass and growth, comprises collection of ovarian follicular fluid from a mammal and at least partially purifying said material. Another method for purifying Micrin comprises collection of blood from a mammal and at least partially purifying said material, wherein during the collection a chelating agent is mixed with the collecting blood. Micrin or Micrin blockade has utility in the treatment of hyperplastic disorders, e.g. cancer, and also for preventing and treating a tissue-deficit disorder, or for controlling the shape, appearance or density of cells and tissues.

### FIELD OF THE INVENTION

The current invention relates to a purified material which generally reduces the size and growth of cells and tissues in animals, and which may have other endocrinological and physiological effects.

### BACKGROUND OF THE INVENTION

WO-A-00/32208 discloses an isolated material having an anti-organotrophic effect (“Micrin”). Micrin was derived from venous plasma by treating sheep with clomiphene, a known synthetic oestrogen receptor ligand. Fractions were derived from the plasma by size-exclusion membrane purification followed by gel filtration and ion-exchange chromatography. Fractions obtained from this process were shown to reduce the masses of internal organs in male and female rats.

Micrin is part of the “organotrophic system”, the totality of up-regulatory and down-regulatory homeostatic influences which determine internal tissue and organ size. These influences are endocrinological (e.g. growth hormone, insulin, the sex steroids, angiogenic factors) and of other kinds (e.g. neurophysiological and immunological). Among the endocrinological influences is Micrin, an endogenous hormone that has the ability to reduce the size of internal organs. It is produced by the ovaries and testes of mammals and other multicellular organisms and probably by non-gonadal tissues as well. Micrin has size-reducing effects encompassing intracrine, autocrine, paracrine (e.g. anti-ovariotrophic action) and endocrine (e.g. anti-hypophysiotrophic action) aspects.

### SUMMARY OF THE INVENTION

The present invention is based on (a) an improved production of Micrin, (b) the mechanisms of action of Micrin, and (c) an extension of the potential uses of Micrin to novel areas of therapy and to diagnosis.

One improvement in the production process involves the use of a chelating agent during blood collection, to stabilise the active moiety. The preferred chelating agent is ethylenediaminetetraacetic acid (EDTA). EDTA has been shown to be preferable to the previously used heparin.

It has also been found that the production process can usefully involve collection of blood from aged female sheep, preferably in the latter half of their life expectancy, rather than from younger animals. Collection of blood is preferably during the follicular phase of the oestrous cycle, rather than at ovulation or during the luteal phase. It is also preferable to monitor the purification process by an assay of apoptosis, using bone marrow stem cells in vitro.

A preferred novel starting material is ovarian follicular fluid from, for example, sheep, pig or cow. For Micrin, a potent cellular inhibitor and apoptotic agent, to be present in high concentration in the fluid bathing the ovum is unexpected.

Micrin has been shown to induce cell shrinkage, reduce cell numbers by apoptosis, inhibit angiogenesis and reduce the dimensions and mass of internal organs and tissues. These effects have been demonstrated in vitro in normal cells (e.g. bone marrow stem cells) and in cancer cells (e.g. prostate and breast), and in vivo using whole animals and surgically altered animals.

Other mechanisms of action may be involved, such as inhibition of cell division (particularly, but not exclusively, at the stage of DNA synthesis), alterations in intracellular micro-architecture and changes in cell-cell adhesion dynamics.

It is believed that Micrin may be peptidic in character.

It is believed that Micrin is produced by the testes and ovaries, though other sources of production in vivo are probable.

Cross-species activity has been demonstrated by administering micrin derived from sheep to rats and causing organ shrinkage. Micrin from ovine and bovine sources has also been shown to be active in cultures of rat cells in vitro, while ovine material has been shown to be active against human cell lines. In addition, Micrin from bovine sources has been shown to inhibit the compensatory renal growth which follows unilateral nephrectomy, this experiment also demonstrating cross-sex activity, in that Micrin from female animals is active in males.

No overt toxic effects have been noted in vivo.

The measurement of Micrin levels in the circulation or at its sites of action will have applications in research and diagnosis (e.g. benign prostatic hyperplasia, osteoporosis).

The material has novel potential therapeutic applications in cancers of the prostate, breast and other tissues, Paget's disease of bone, acromegaly, uterine fibroids and hepatomegaly, among other disorders.

The blockade of the material's activity, via antibodies, receptor antagonists or other forms of inhibitor, can be envisaged in the treatment of postmenopausal osteoporosis and other tissue-deficit disorders.

The material and blockade of the material can be envisaged to form the basis of novel veterinary therapeutics and agents for use in horticulture and agriculture.

The measurement of the material will be of value in clinical and veterinary diagnosis and research, and in horticultural and agricultural applications.

### BRIEF DESCRIPTION OF THE FIGURES

FIG. 1 shows survival of bone marrow stem cells in an apoptosis assay in vitro after exposure to various plasmas harvested from sheep blood samples collected using heparin or EDTA.

FIG. 2 shows lower organ weights among rats receiving the HPLC eluate from the plasma of ovary-intact sheep plasma versus rats receiving the corresponding material from ovariectomized sheep.

FIG. 3 shows survival of bone marrow stem cells in an apoptosis assay in vitro of individual HPLC fractions.

FIG. 4 shows a dose-response curve for an HPLC ion exchange fraction highly apoptotic in vitro.

FIG. 5 shows shrinkage of bone marrow stem cells in vitro, exposed to plasma from ovary-intact and ovariectomised sheep, versus saline controls.

FIG. 6 shows induction of apoptosis in human prostate cancer cells in vitro.

FIG. 7 shows induction of apoptosis in human breast tumour cells in vitro.

FIG. 8 shows inhibition of the compensatory renal growth which follows unilateral nephrectomy, after the administration of ion exchange fractions of bovine ovarian follicular fluid.

### DESCRIPTION OF THE INVENTION

Blood for the production of Micrin is preferentially obtained from aged sheep (above, for example, seven years of age) in the follicular phase of the oestrous cycle. Luteal phase blood from aged sheep also contains Micrin, but usually at a lower concentration. Micrin is at low concentration in the blood of lambs.

A richer source of Micrin is ovarian follicular fluid, unexpectedly so because of the adjacency of high concentrations of this inhibitor to the developing ovum. Ovarian follicular fluid may be obtained from the abattoir-derived ovaries of sheep, cows or pigs.

With reference to the five-step process defined below, Micrin can be purified using membrane filtration, followed by gel filtration and high performance liquid chromatography (HPLC) ion exchange column chromatography. Micrin activity can be assessed by the suppression of cell viability in vitro (e.g. using the BMSC assay given as Example 4 below.)

In addition to purification from natural sources, Micrin may also be prepared by synthesis or recombinant DNA methods.

Micrin causes apoptosis of prostate and breast tumour cells in vitro. That transformed cells respond in the same way to Micrin as normal cells was unanticipated. Potential novel therapeutic applications for Micrin thus include the prevention and treatment of cancers of the prostate, breast and other tissues. Other potential therapeutic applications are bone overgrowth disorders such as Paget's disease and fibrodysplasia ossificans progressive, bone metastases, hepatomegaly, splenomegaly, uterine fibroids and other female and male reproductive system ailments, pregnancy complications, acromegaly, paediatric and adolescent tissue overgrowth disorders, obesity, breast reduction, skin and other external organ conditions, and immune system disorders.

Micrin, delivered by whatever route of administration, may be used alone therapeutically or in conjunction with other agents For example, in benign prostatic hyperplasia, treatment with an inhibitor of androgen synthesis might accompany Micrin therapy.

As indicated above, Micrin blockade is of value in therapy. For this purpose, the active agent may be, for example, an antibody. A suitable monoclonal or polyclonal antibody to Micrin may be raised and used by means known to those of ordinary skill in the art.

Potential novel therapeutic applications for Micrin blockade (achieved by whatever means) include the prevention and treatment of postmenopausal osteoporosis, growth disorders, paediatric and adolescent tissue undergrowth disorders, reproductive disorders and pregnancy complications, skin and other external organ conditions, breast augmentation, and neurodegenerative disorders.

Micrin blockade, delivered by whatever route, may be used alone therapeutically or in conjunction with other agents. For example, in post-menopausal osteoporosis, oestrogen treatment may accompany Micrin blockade.

The use of Micrin and Micrin blockade is envisaged to be useful not only in controlling the size of cells and tissues but in controlling their shape and appearance, for example in wound healing and in cosmetic and reconstructive surgery.

The use of Micrin and Micrin blockade is envisaged to be useful not only in controlling the size of cells and tissues and their size and appearance but in controlling their density, for example in conditions affecting bone.

Measurements for diagnosis and research will be of value in conditions of Micrin excess and deficiency. In benign prostatic hyperplasia, for example, a deficiency in Micrin may be involved in causation, implying that Micrin treatment of this condition can be represented as hormone replacement for men. In postmenopausal osteoporosis, in contrast, an excess of Micrin may be involved in causation, as levels of the countervailing organotrophic agent, ovarian oestrogen, fall.

Novel veterinary therapeutics can be envisaged on the basis of the administration of Micrin and the blockage of Micrin (achieved by whatever means), by whatever route of administration and administered alone or in combination (for example, diseases equivalent to those seen in humans such as benign and malignant diseases of the prostate and breast, and applications with no human equivalent, such as the control of antler growth).

Measurement of Micrin levels can be envisaged as being useful in veterinary diagnosis and research.

Novel agents for use in horticulture and arable farming can be envisaged on the basis of the administration of Micrin and the blockage of Micrin (achieved by whatever means), by whatever route of administration and administered alone or in combination. An example might be the restriction of tree growth. Measurement of Micrin levels can be envisaged as being useful in the context of horticulture and arable farming.

As Micrin may be peptidic in character, small molecule agonists and antagonists are envisaged.

The activity of clomiphene, an established ligand of the oestrogen receptor, as a small molecule inducer of Micrin, is disclosed in WO-A-00/32208 (the content of which is incorporated herein by reference). The ability to induce Micrin is envisaged to be a property of other compounds similar to clomiphene in biological or chemical terms and also a property of compounds which are dissimilar.

PROTOCOL

1A. Blood collected over EDTA is centrifuged at +4° C. and 2000 g for 10 minutes.

1B. Ovarian follicular fluid obtained by aspiration of abattoir-derived ovaries is centrifuged at +4° C. and 2000 g for 10 minutes.

2. Plasma or ovarian follicular fluid spun through Amicon Centriprep-30 cartridges at 1800 g to give a nominal 0-30 kD fraction.

3. Nominal 0-30 kD fraction spun through Amicon Centriprep-3 to give a nominal 3-30 kD sub-fraction.

4. Nominal 3-30 kD fraction concentrated and gel filtered through a Pharmacia Superdex-75 column to give a nominal 10-20 kD sub-fraction.

5. Nominal 10-20 kD sub-fraction was diluted with buffer and concentrated and applied to a Vydac's Protein SAX HPLC ion exchange column and eluted with a gradient of 0-1M NaCl, fractions being collected.

The presence and level of activity of Micrin at any stage can be confirmed by the in vitro assay reported below as Example 4, as well as by the rat bioassay reported below as Example 3 and the renal hypertrophy inhibition assay as Example 5. Alternatively, mass spectrometry can be used or other analytical methods such as gel electrophoresis or liquid chromatography.

Micrin-rich biological fluids may be collected from an animal such as the sheep. Ovarian lymph or ovarian venous plasma may be collected, following the method of Heap et al. J. Reprod. Fert. (1985), 74: 645-656, or, more conveniently, jugular vein plasma may be used. Blood collected during the follicular phase of the oestrous cycle tends to show greater Micrin activity than that collected during the luteal phase. A particularly rich source of Micrin is ovarian follicular fluid itself.

### EXAMPLE 1

Preparation of Micrin

1A. Collection of Sheep Plasma

Multiparous non-pregnant ewes were used, aged 7-13 years. Ewes were housed with a vasectomised ram to detect the day of oestrus (Day 0). Luteal phase blood was taken on Day 6, follicular phase blood on Day 15 post-oestrus. Blood was collected without anaesthesia from the jugular vein, onto EDTA (1 mg/ml blood). Blood samples were stored on ice prior to centrifugation. The plasma was then obtained from the blood by centrifugation at +4° C. and 2000 g for 10 minutes. The plasma layer was then removed and stored in a plastic bottle in a freezer at −20° C.

1B. Collection of Ovarian Follicular Fluid

Ovaries were collected post-mortem from abattoir sheep, cows and pigs, and stored on ice. Fluid was aspirated from the ovarian follicles using a standard hypodermic syringe. The ovarian follicular fluid was then centrifuged at +4° C. and 2000 g for 10 minutes, prior to storage in a freezer at −20° C.

2. Preparation of the Nominal 0-30 kD Molecular Weight Fraction

The plasma (120 ml) or ovarian follicular fluid (120 ml) was dispensed into 8 Amicon Centriprep-30 filtration units, a centrifuge device with concentric inner and outer compartments separated by a membrane having a nominal cut off size of 30 kD. The device was spun at 1800 g for 14-24 hours at +4° C. The filtrate was harvested at intervals and centrifugation continued until a final volume of 80 ml was obtained. This nominal 0-30 kD fraction was stored in polypropylene tubes at −20° C.

3. Preparation of the Nominal 3-30 kD Molecular Weight Sub-Fraction

The nominal 0-30 kD fraction, from 2. above, was dispensed into 6 Amicon Centriprep 3 filtration units and centrifuged at 1800 g. for 5-6 hours at +4° C. Centrifugation was performed until 2 ml of retentate remained in the outer compartment of each Centriprep 3 unit. The retentate (3-30 kD molecular weight sub-fraction) was stored overnight at −10° C.

The retentate was subsequently concentrated to a final volume of 500 μl in an Amicon Centricon 3 unit. After centrifuging the samples at 2800 g for 2-4 hours at +4° C. the retentate was placed on ice before applying to the gel filtration column as detailed below.

4. Preparation of the Nominal 10-20 kD Molecular Weight Sub-Fraction

Samples (2×200 μl) prepared as in 3. above were subsequently applied to an FPLC gel filtration column (Pharmacia Superdex-75, HR 10/30, 30 cm long, 1 cm diameter) that had been calibrated with protein molecular weight standards. Elution was effected in phosphate-buffered saline and fractions (1 ml/tube) were collected over a period of 45 minutes.

Fractions falling within the nominal 10-20 kD molecular weight range were pooled and concentrated to 2 ml by centrifugation in an Amicon Centriprep 3 unit (1800 g. for 1-2 hours at +4° C.). The retentate was either stored in polypropylene tubes. at −20° C. or used in 5. below.

5. Preparation of the Ion Exchange (Sodium Chloride) Fractions from the Nominal 10-20 kD Molecular Weight Sub-Fraction

The nominal 10-20 kD molecular weight sub-fraction, from 4, above, was subjected to buffer exchange by dilution to 15 ml in 20 mM Tris HCl buffer, pH 7.6. The Centriprep 3 unit was centrifuged as before (1800 g at +4° C., 6-8 hours). This fraction was further concentrated to 500 μl in Amicon Centricon 3 units (3000 g for 1-2 hours at +4° C.).

Samples (2×200 μl) were then applied to a Vydac's Protein SAX HPLC ion exchange column (0.75×5 cm) and eluted with a linear gradient of 0-1M sodium chloride in 20 mM Tris HCl buffer, pH 7.6. Eluted fractions (2 ml/tube) were collected over 45 minutes and their activities tested in in vitro cell assays and in in vivo rat bioassays, as well as by mass spectrometry and other analytical methods.

### EXAMPLE 2

Pharmaceutical Composition

Micrin for therapeutic applications is obtained by putting 120 ml of follicular phase sheep plasma or ovarian follicular fluid through the production method described in Example 1. HPLC fractions are obtained having maximal apoptotic activity in the in vitro assay described in Example 4. These fractions may be used as a pharmaceutical or they may be sterile filtered, lyophilized and reconstituted with physiological, pyrogen-free saline and supplied in a sterile glass vial. The dose (for example 5 ml) can be administered by injection using a sterile needle.

### EXAMPLE 3

In Vivo Bioassay of Micrin

The presence of Micrin in fractions from Example 1 can be demonstrated using the in vivo bioassay described below:

Adult female Sprague Dawley rats (weight 200-220 gm) or adult male Wistar albino rats (weight 300-400 gm) were injected intraperitoneally using sterile needles, 0.5×16 mm, 25G, with daily 1×1 ml doses of the sheep plasma or plasma fraction for four days. Ninety six hours after the commencement of dosing, the rats were then anaesthetised (terminally using carbon dioxide) and the thorax opened surgically. The rats were then partially exsanguinated by cardiac puncture (5 ml); and then each of the rats underwent whole body dissection as described by Hart in Toxicology (1990), 61: 185-194.

The following organs were removed from the partially exsanguinated female rats in a standard order, trimmed free of connective tissue and fat, and weighed: heart, liver, pituitary gland, adrenal glands, kidneys, spleen, uterus and ovaries; from male rats: heart, liver, pituitary gland, adrenal glands, kidneys, spleen, prostate and testes. Each of the organs was weighed and the results expressed as a proportion of the whole rat terminal body weight (gm/kg or %).

Control plasma experiments were carried out using the same procedure with sheep jugular whole venous plasma and similar fractions (molecular weight or ion exchange fractions) derived from sheep jugular venous plasma obtained as above in Example 1, but from ovariectomised sheep. Control experiments for ovarian follicular fluid used saline or compared each fraction (for example from ion exchange chromatography) with the others.

### EXAMPLE 4

In Vitro Bioassay of Micrin

The presence of Micrin is demonstrated using a bone marrow stem cell (BMSC) assay, of the kind described in Scutt and Bertram, J. Bone and Mineral Res (1995), 10: 474-489. This assay is carried out using BMSCs isolated as in a) below.

4a) Isolation of Rat Bone Marrow Cells

Rats were killed by cervical dislocation, or other “Schedule 1” method.

Before dissection, the rats were sprayed with 70% Industrial Methylated Spirit (IMS) to kill fungal spores etc.

The back legs were removed cleanly at the hip joint.

Under sterile conditions, all soft tissue was removed and the bones separated.

The growth plates were removed from both the femur and the tibia.

Using a circular saw, the tibia was cut at the tibial/fibula junction and the femur a little below the hip joint.

The cut bones were placed, cut side down, in inserts in eppendorf tubes and spun at 2000 rpm for 1 min. in a centrifuge. As a result all of the bone marrow was then deposited in the bottom of the tube. The bones and inserts were removed.

4b) Preparation of Cell Culture

A cell culture medium was prepared, consisting of:

500 ml DMEM with non-essential amino acids and 4500 mg/l glucose

5 ml Glutamax

5 ml Pen/Strep

5 ml pyruvate

25 mg ascorbic acid, final concentration of circa 50 mg/ml

70 ml foetal calf serum, final conc. 12% v/v

The rat bone marrow from 4a above was resuspended in 0.5 ml culture media (“BJND”) and then made up to a volume of 5 ml per bone in BJND.

A single cell suspension was then made by forcing the resuspended bone marrow through an 18G needle and made up to a volume of 10 ml per bone with BJND. The cells were counted using a haemocytometer and the final cell density adjusted to 4×10⁶ mononuclear cells/ml.

4c) Measurement of Cell Viability by Alamar Blue

BMSC's were obtained as in 4a above.

Cells were plated out in culture flasks with a surface area of 75 cm²), approx. 1 leg's worth of cells per flask in BJND, 4b above.

After 5 days, this was split 50:50 into new flasks still in BJND.

When confluent, the cells were subcultured into 24 well plates at a density of 25,000 cells per well in 0.3 ml BJND, allowing for “no control”, “positive control” and “no cell control”.

These were left overnight to attach and the next day fractions for testing were added.

After 24 hours 30 μl Alamar blue (source Serotec) was added and the cultures left to metabolise for 4 hours.

200 μl aliquots were removed and the optical absorption measured in a photometer at 650 nm.

Results were calculated as follows:

(experimental−no cell control)/(no drug control−no cell control)×100. This yields results as a percent control viability.

### EXAMPLE 5

In Vivo Bioassay of Micrin Using Inhibition of Compensatory Renal Growth

The presence of Micrin in fractions from Example 1 can be demonstrated using the in vivo bioassay described below, involving compensatory renal growth. An example of the use of this kind of assay is given in Haylor et al, J. Am. Soc. Nephrol. (2000) 11: 2027-2035.

Male Wistar rats (250-350 gm) were subjected to left nephrectomy through a left flank incision, after induction of anaesthesia with halothane, and the excised kidney was weighed. Ion exchange fractions from Example 1 above were infused continuously into the pelvic region of the remaining kidney, via a perforated catheter, using an osmotic mini-pump (Alza). After 7 days, the remaining kidney was removed and weighed. The effects of different fractions on compensatory renal growth were compared, via measurements of kidney wet and dry weight and whole kidney protein and DNA. Kidney sections were analysed for apoptosis using Apoptag staining.

Referring now to the drawings:

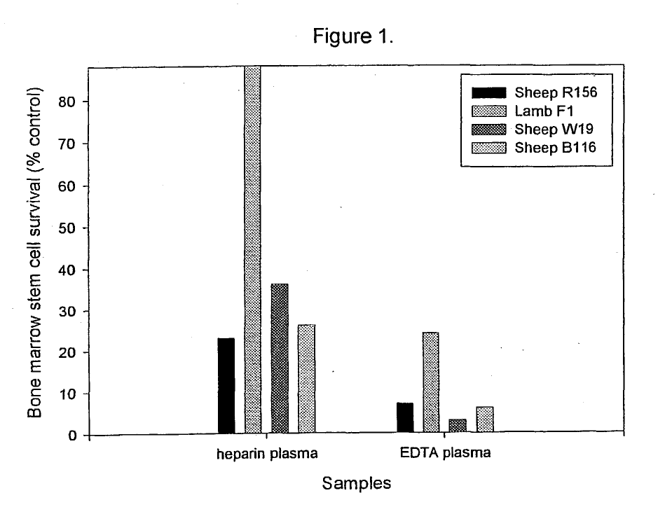

FIG. 1 illustrates an assay in vitro using bone marrow stem cells, in which an apoptotic effect on cell viability is seen most strongly in sheep plasma containing with EDTA, as compared with heparinised plasma. (R156, W19 and B116 denote samples from individual sheep over seven years old, F1 denotes a sample from a lamb);

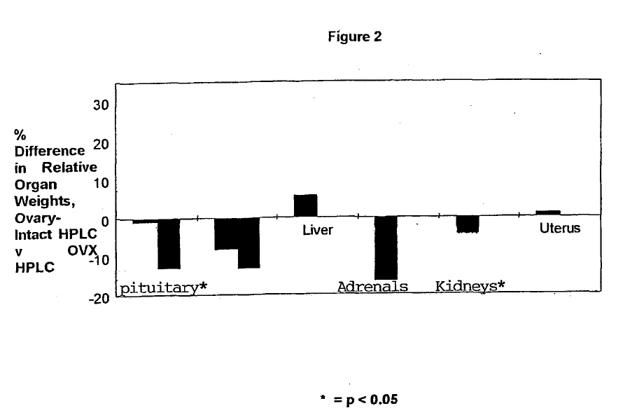

FIG. 2 shows the results of a rat bioassay (n=7) of sheep plasma subjected to membrane and gel filtration and HPLC ion exchange chromatography, the HPLC eluate in total from an ovary-intact sheep being compared with the HPLC eluate deriving from plasma from ovariectomised sheep, with organ weights being lower in the rats receiving ovary-intact sheep material;

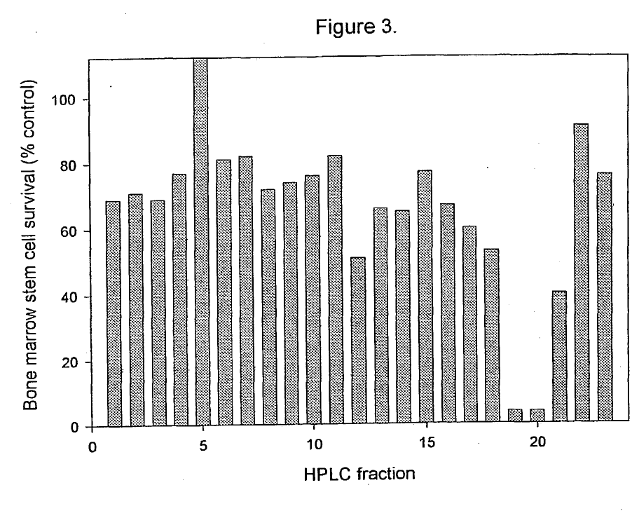

FIG. 3 illustrates the in vitro assay using bone marrow stem cells of HPLC ion exchange fractions produced from ovary-intact sheep, showing greatest apoptotic activity in fractions 18-20;

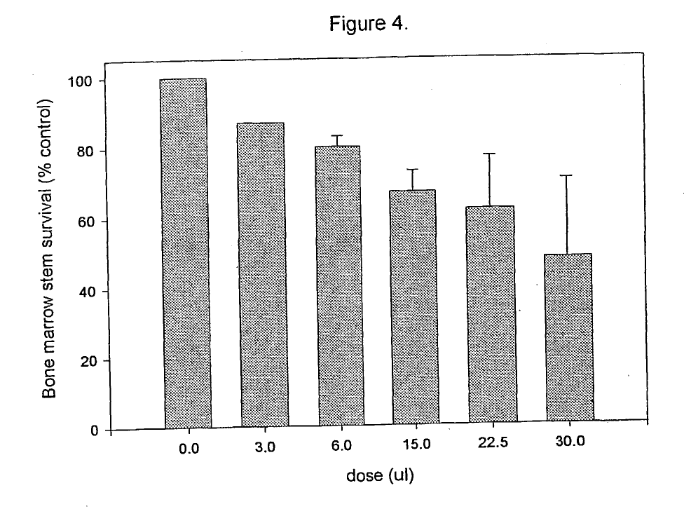

FIG. 4 illustrates a typical dose-response curve for an HPLC fraction, highly apoptotic in vitro, of membrane-filtered and gel-filtered plasma;

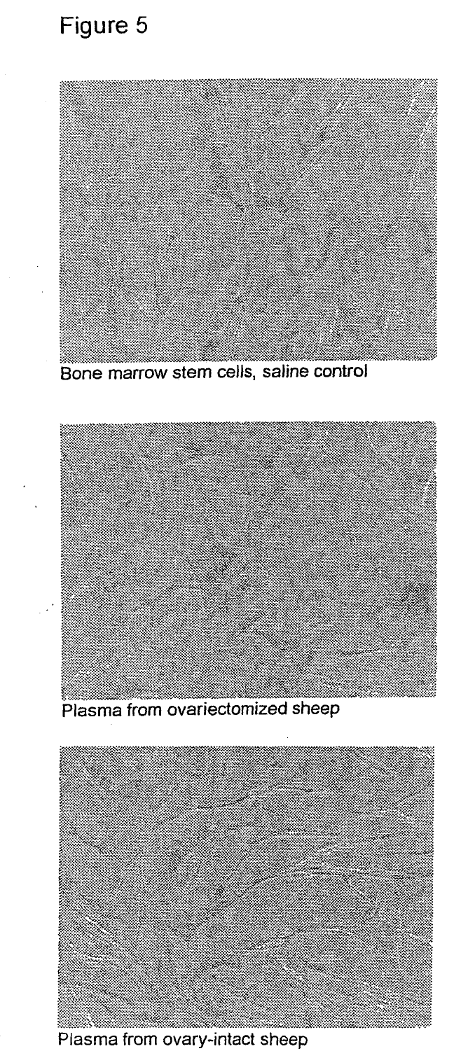

FIG. 5 illustrates cell shrinkage of bone marrow stem cells in vitro, with cells exposed to saline, ovariectomised sheep plasma and ovary-intact sheep plasma:

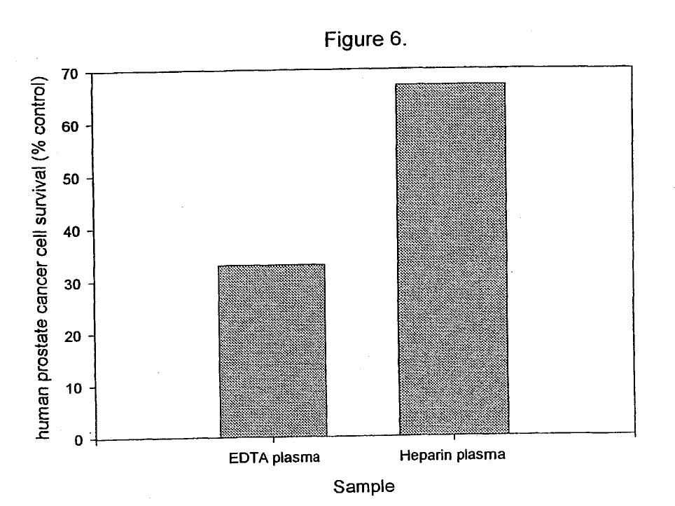

FIG. 6 illustrates the induction after 24 hours of apoptosis in vitro in human prostate cancer cells of the DU145 cell line by plasma (EDTA and heparin) from the same sheep blood sample, the plasma having previously proved highly active in an in vitro assay of apoptosis involving bone marrow stem cells;

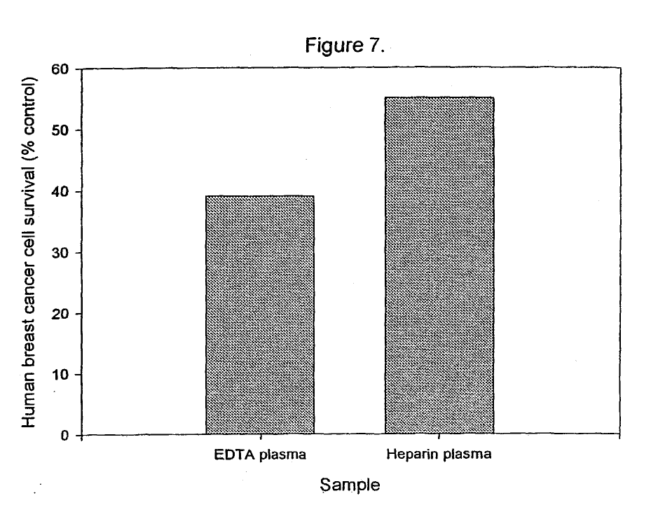

FIG. 7 illustrates the induction after 24 hours of apoptosis in vitro in human breast tumour cells of the MC6 cell line by plasma (EDTA and heparin) from the same sheep blood sample, the plasma having previously proved highly active in an in vitro assay of apoptosis involving bone marrow stem cells.

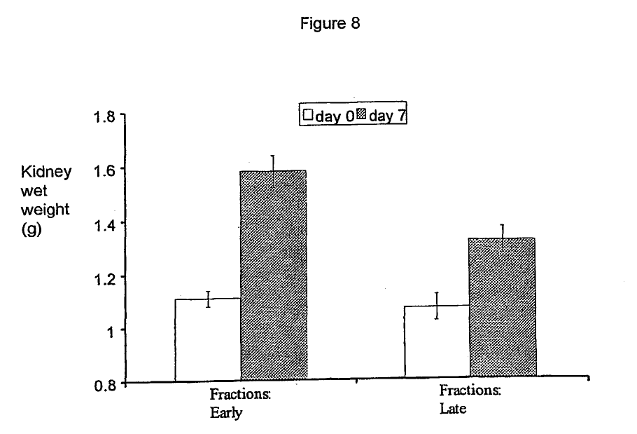

FIG. 8 illustrates the alternative in vivo bioassay which involves the inhibition of compensatory renal growth in the unilaterally nephrectomised male rat. The “Day 0” wet kidney weight in grams is the mean (n=4) weight of the excised organ; the “Day 7” kidney weight is the weight of the remaining organ after the infusion of ion exchange fractions of ovarian follicular fluid. Here, the administration of one pair of fractions from early in an ion exchange run is associated with a 45% lower level of compensatory renal growth than is seen after the administration of another pair of fractions from later in the same ion exchange run. The difference was significant, p<0.005.

The results shown in FIG. 5 may be tabulated as follows:

| | Cell cross-sectional area, |
|---|---|
| Bone marrow stem cells in vitro | mean ± SD |
| 1. Saline control | 0.670 ± 0.382 |
| 2. Plasma from ovariectomised sheep | 0.779 ± 0.547 |
| 3. Plasma from ovary-intact sheep | 0.467 ± 0.233 |

1. A method of purifying a material (“Micrin”) with the ability to reduce cell, tissue and organ size, mass and growth, the method comprising collection of ovarian follicular fluid from a mammal and at least partially purifying said material.

2. A method of purifying a material (“Micrin”) with the ability to reduce cell, tissue and organ size, mass and growth, the method comprising collection of blood from a mammal and at least partially purifying said material, wherein during the collection a chelating agent is mixed with the collecting blood.

3. A method as claimed in claim 2, wherein the chelating agent is EDTA.

4. A method as claimed in claim 2 or claim 3, wherein the blood is collected from an aged female mammal.

5. A method as claimed in any of claims 2 to 4, in which the mammal is female and the blood is collected during the follicular phase of the oestrous cycle.

6. A method as claimed in any of claims 1 to 5, in which the purification process is monitored by assay of apoptosis in vitro.

7. A method as claimed in any of claims 1 to 5, in which the purification process is monitored by in vivo bioassay involving alterations in organ size.

8. A method of making a material (“Micrin”) with the ability to reduce cell, tissue and organ size, mass and growth, the method comprising collecting blood from a mammal and harvesting plasma from it, purifying by molecular sizing, followed by ion exchange chromatography, wherein the elution process uses a salt solution of increasing concentration up to 1M, and selecting fractions that are active in an assay of apoptosis in vitro or an in vivo bioassay involving alterations in organ size.

9. A method of making a material (“Micrin”) with the ability to reduce cell, tissue and organ size, mass and growth, the method comprising purifying ovarian follicular fluid by molecular sizing, followed by ion exchange chromatography, wherein the elution process uses a salt solution of increasing concentration up to 1M, and selecting fractions that are active in an assay of apoptosis in vitro or an in vivo bioassay involving alterations in organ size.

10. Use of Micrin, obtainable by a method as claimed in any of claims 1 to 9, for the manufacture of a medicament for the prevention and treatment of a hyperplastic disorder.

11. Use of an antibody-to Micrin or another agent causing blockade of Micrin, for the manufacture of a medicament for preventing or treating a tissue-deficit disorder, or for controlling the shape, appearance or density of cells and tissues.

12. Use as claimed in claim 10, for the treatment of cancer.

13. Use as claimed in claim 11, for the treatment of postmenopausal osteoporosis.

14. Use as claimed in claim 10 or claim 11, for use in wound healing, cosmetic or reconstructive surgery or a condition affecting bone.
